(19) United States
(12) Patent Application Publication
(10) Pub. No.: US 2017/0202444 A1
(43) Pub. Date: Jul. 20, 2017

(54) ENDOSCOPE AUXILIARY DEVICE

(71) Applicants: BOE TECHNOLOGY GROUP CO., LTD., Beijing (CN); BOE OPTICAL SCIENCE AND TECHNOLOGY CO., LTD., Suzhou, Jiangsu (CN)

(72) Inventor: Chunlei CAO, Beijing (CN)

(21) Appl. No.: 15/100,097

(22) PCT Filed: Oct. 21, 2015
(86) PCT No.: PCT/CN2015/092412

§ 371 Date: May 27, 2016

(30) Foreign Application Priority Data
Jun. 19, 2015 (CN) 201510345139.4

(51) Int. Cl. A61B 1/06; A61B 5/00; A61B 5/01; A61B 1/04; A61B 1/00

(52) U.S. Cl. CPC A61B 1/0684; A61B 1/04; A61B 1/00045; A61B 5/01; A61B 5/746

(57) ABSTRACT

An endoscope auxiliary device is provided, which can reduce light spot and light shadow formed on an object and improve an accuracy of a result of an endoscope observation. The endoscope auxiliary device includes an operating handle and a probing body connected with each other, wherein, a switching device is provided on the operating handle. Optical mesh points are provided on a surface of the probing body, and a light source controlled by the switching device and a reflective housing for reflecting light of the light source into the probing body are provided on an end of the probing body dose to the operating handle.

### TECHNICAL FIELD

Embodiments of the present disclosure relate to an endoscope auxiliary device.

### BACKGROUND

Ear cleaning is a regular behavior in our daily life. A conventional ear picker is mainly made of metals, plastics, woods or bamboos. Ear canal is tortuous and deep, light cannot irradiate to the ear canal, so that it is very inconvenient to clear earwax.

In a conventional ear picker, the inconvenience described above is resolved to a certain degree by providing a light source on the ear picker, however, there is insufficient utilization ratio of illumination of the light source. So it is easy to form defects, such as light spot, light shadow and others on the object, and the observing result is affected thereby; while, if a light source of high brightness is used, a light spot may be formed, and the high brightness may be harm to the eyes of a user.

### SUMMARY

Embodiments of the present disclosure provide an endoscope auxiliary device, including an operating handle and a probing body connected with each other.

A switching device is provided on the operating handle.

Optical mesh points are provided on a surface of the probing body. A light source controlled by the switching device and a reflective housing for reflecting light of the light source into the probing body are provided on an end of the probing body close to the operating handle.

In an example, the light source is embedded in an end of the probing body close to the operating handle, the reflective housing is provided on the surface of the end of the probing body close to the operating handle, and the reflective housing and the end of the probing body close to the operating handle arc fixed in the operating handle.

In an example, a magnifying device is provided on an end of the probing body away from the operating handle.

In an example, the light source includes a light-emitting diode, and the probing body includes a probing body made of material of polymethyl methacrylate.

In an example, the endoscope auxiliary device includes an endoscope auxiliary device for nasal cavity, an endoscope auxiliary device for ear canal, a laiyngendoscope auxiliary device, or an endoscope auxiliary device for mouth.

In an example, a health information collecting probe configured to collect health information of an object is provided on an end of the probing body away from the operating handle; and a controller signally communicated with the health information collecting probe and a display device signally communicated with the controller are provided on the operating handle, the controller being configured to perform data processing on the health information of the object, and sending the health information of the object to the display device for displaying.

In an example, the controller is configured to trigger the display device to display an alarm when the health information of the object does not meet predetermined conditions.

In an example, an alarm is further provided on the operating handle, the controller being configured to trigger the alarm to give an alarm when the health information of the object does not meet predetermined conditions

In an example, the health information collecting probe includes a temperature collecting probe, a humidity collecting probe or a humiture collecting probe.

In an example, an image collecting device is further provided on an end of the probing body away from the operating handle; and the controller is further signally communicated with the image collecting device, the controller being configured to perform data processing on the image collected by the image collecting device, and sending the image to the display device for displaying.

In an example, at least one of a bluetooth transmission device, a USB interface and a video transmission interface is further provided on the operating handle.

### BRIEF DESCRIPTION OF THE DRAWINGS

Embodiments of the present disclosure will be described in detail hereinafter in conjunction with accompanying drawings to allow one of ordinary skill in the art to understand the present disclosure more clearly, in which:

FIG. 1 is a structural schematic diagram of an endoscope auxiliary device of an embodiment of the present disclosure;

FIG. 2 is a structural schematic diagram of a control system of the endoscope auxiliary device of an embodiment of the present disclosure.

### DETAILED DESCRIPTION

The technical solutions of the embodiments will be described in a clearly and fully understandable way in connection with the drawings related to the embodiments of the disclosure. Apparently, the described embodiments are just a part but not all of the embodiments of the disclosure. Based on the described embodiments herein, those skilled in the art can obtain other embodiment(s), without any inventive work, which should be within the scope of the disclosure.

Unless otherwise defined, all the technical and scientific terms used herein have the same meanings as commonly understood by an ordinary skill in the art to which the present invention belongs. The terms, such as “first,” “second” or the like, which are used in the description and the claims of the present application, are not intended to indicate any sequence, amount or importance, but for distinguishing various components. Also, the terms, such as “a/an,” “the” or the like, are not intended to limit the amount, but for indicating the existence of at lease one. The terms, such as “comprise/comprising,” “include/including” or the like, are intended to specify that the elements or the objects stated before these terms encompass the elements or the objects and equivalents thereof listed after these terms, but not preclude other elements or objects. The terms, such as “On,” “under,” or the like, are only used to indicate relative position relationship, and when the position of the object which is described is changed, the relative position relationship may be changed accordingly.

To reduce light spot and light shadow formed on an object, and improve an accuracy of a result of an endoscope observation, an embodiment of the present disclosure provides an endoscope auxiliary device. To make the objective, the technical solution and the advantages of the present disclosure clearer, the present disclosure shall be further described in detail hereinafter in conjunction with the examples.

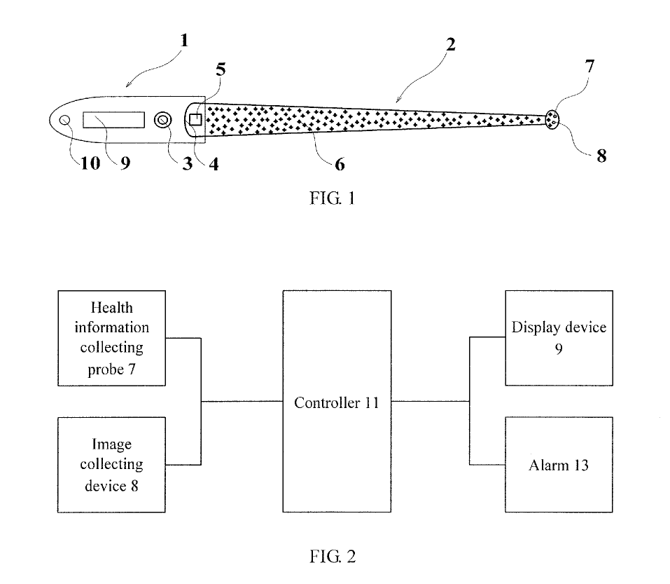

As shown in FIG. 1, an endoscope auxiliary device provided by the embodiment of the present disclosure includes an operating handle 1 and a probing body 2 connected with each other. A switching device 3 is arranged on the operating handle 1. Optical mesh points 6 are provided on a surface of the probing body 2. A light source 5 controlled by the switching device 3 and a reflective housing 4 for reflecting light of the light source 5 into the probing body 2 are arranged on an end of the probing body 2 close to the operating handle 1.

A type of the endoscope auxiliary device may be, but not limited to, for example, such as an endoscope auxiliary device for nasal cavity, an endoscope auxiliary device for ear canal, a laryngendoscope auxiliary device, or an endoscope auxiliary device for mouth. The endoscope auxiliary device shown in FIG. 1, for example, is an endoscope auxiliary device for ear canal, which has a function of ear picker. An ear spoon 12 is arranged on an end of the probing body 2 away from the operating handle 1.

A light-emitting diode has advantages, such as energy-saving, high brightness, low heat, soft light and the like, for example, the light source 5 adopts a light source of the light-emitting diode. Structures of the reflective housing 4, the probing body 2 and the light source 5 are not limited. As shown in FIG. 1, in the embodiment, the light source 5 is embedded in an end of the probing body 2 close to the operating handle 1, and the reflective housing 4 is arranged on a surface of the end of the probing body 2 close to the operating handle 1. The reflective housing 4 and the end of the probing body 2 close to the operating handle 1 are fixed in the operating handle 1. The reflective housing 4 usually includes a base layer and a reflective layer. The reflective layer is arranged to face the light source 5, and the reflective layer may effectively reflect light of the light source 5 into the probing body 2.

For example, the probing body 2 is made of material of polymethyl methacrylate; luminance of the light source may be improved by using the material of polymethyl methacrylate, and light scattering may be suppressed, so that a utilization ratio of the light source may be improved. The light spot and light shadow formed in the ear canal may be reduced, and the probing body made of the material of polymethyl methacrylate has good toughness, which may not be easily broken, so as to reduce risk of breaking at the time of cleaning ear.

In an embodiment of the present disclosure, the reflective housing 4 may reflect light emitted by the light source 5 into the probing body 2, and the reflected light is scattered in various directions by using an optical mesh point 6 on the surface of the probing body 2 and conditions of total reflection is destroyed, so that the emitted light is more uniform, and a utilization ratio of the light source may be improved. The sufficient light is irradiated onto the object, and the light spot and light shadow formed on the object are reduced, as well as an accuracy of a result of an endoscope observation is improved. By using the endoscope auxiliary device shown in FIG. 1, the ear canal is probed and the earwax is cleaned, and the observation and operation are more convenient and accurate.

In other embodiments of the present disclosure, for example, for an endoscope auxiliary device for oral cavity, a magnifying glass may be arranged on an end of the probing body away from the operating handle. When a mouth is observed by using the mouth endoscope, the magnifying glass may magnify a part to be observed in the mouth, so that a user may observe the part to be observed in the mouth more clearly.

For example, refer to FIG. 1 and FIG. 2 again, a health information collecting probe 7 may be provided on an end of the probing body 2 away from the operating handle 1, and a controller 11 signally communicated with the health information collecting probe 7 is arranged on the operating handle 1, and the controller 11 performs data processing on the health information, and sends the health information to the display device 9 for displaying. When probing the ear canal or cleaning the earwax, the health information collecting probe 7 accurately monitors the health information of the ear canal in real time, and sends the health information to the controller 11. The controller 11 processes and sends the health information to the display device 9 for displaying, so that the user may conveniently and quickly know a health condition of the ear canal by viewing the display device 9.

In the embodiment of the present disclosure, once the health information collecting probe 7 detects abnormality in the health information, for example, exceeding a predetermined value, continuously increasing, continuously decreasing or the like, the controller 11 may trigger the display device 9 to display an alarm. For example, the display device 9 may display messages such as “hyperthermia or higher body temperature”, “higher humidity in the ear canal” or the like, so that the user may be prompted that the ear canal has probably had a pathological change.

The health information collecting probe 7 may be, but not limited to, such as a temperature collecting probe, a humidity collecting probe or a humiture collecting probe. The health information collecting probe 7 may be, but not limited to, a collecting probe of a resistive type, a capacitive type or an infrared type, or the like. In an embodiment of the present disclosure, temperature and/or humidity of the ear canal and the earwax may be monitored in real time by using the health information collecting probe 7. Since the temperature and humidity reflect a result of metabolism of an organism, and a normal temperature and a humidity are necessary conditions for the organism to perform normal functions, a health condition of the ear canal may be better reflected by using the humiture collecting probe.

As further shown in FIG. 1 and FIG. 2, an image collecting device 8 is provided on an end of the probing body 2 away from the operating handle 1, and the controller 11 is signally communicated with the image collecting device 8. The controller 11 performs data processing on the image collected by the image collecting device 8, and sends the image to the display device 9 for displaying. The user may conveniently observe images of an object by viewing the display device 9, which is benefit to accurately determining a probable focus of the object in time.

In an embodiment of the present disclosure, at least one of a bluetooth transmission device, a USB interface and a video transmission device may be further arranged on the operating handle. With those transmission interfaces, data information may be transferred to an external device for further analyzing. For example, in the embodiment shown in FIG. 1, a video transmission interface 10 is arranged on the operating handle 1 of the endoscope auxiliary device.

As shown in FIG. 2, in other embodiments of the present disclosure, an alarm 13 may be further arranged on the operating handle. When an abnormality is presented in the health information collected by the health information collecting probe 7, for example, exceeding a predetermined value, continuously increasing, continuously decreasing or the like, and the control device may trigger the alarm 13 to give an alarm, to prompt the user that the object may have a pathological change.

It is understood that the foregoing embodiments are only exemplary embodiments of the present disclosure to illustrate the principle of the embodiments of the present disclosure; however, the embodiments of the present disclosure are not limited thereto. Any modifications, equivalent alternations and improvements without departing from the spirit and principle of the present disclosure shall fall within the scope of the present disclosure.

The application claims priority of Chinese patent application No. 201510345139.4 filed on Jun. 19, 2015 and entitled “Endoscope Auxiliary Device”, the contents of which are incorporated herein by reference entirely.

1. An endoscope auxiliary device, comprising an operating handle and a probing body connected with each other, wherein,
a switching device provided on the operating handle; and
optical mesh points provided on a surface of the probing body; a light source controlled by the switching device and a reflective housing configured to reflect light of the light source into the probing body being provided on an end of the probing body close to the operating handle.

2. The endoscope auxiliary device according to claim 1, wherein the light source is embedded in an end of the probing body close to the operating handle, the reflective housing is provided on the surface of the end of the probing body close to the operating handle, and the reflective housing and the end of the probing body close to the operating handle are fixed in the operating handle.

3. The endoscope auxiliary device according to claim 1, wherein a magnifying device is provided on an end of the probing body away from the operating handle.

4. The endoscope auxiliary device according to claim 1, wherein the light source includes a light-emitting diode, and the probing body includes a probing body made of material of polymethyl methacrylate.

5. The endoscope auxiliary device according to claim 1, wherein the endoscope auxiliary device comprises an endoscope auxiliary device for nasal cavity, an endoscope auxiliary device for ear canal, a laryngendoscope auxiliary device, or an endoscope auxiliary device for mouth.

6. The endoscope auxiliary device according to claim 1, wherein,
a health information collecting probe configured to collect health information of an object is provided on an end of the probing body away from the operating handle; and
a controller signally communicated with the health information collecting probe and a display device signally communicated with the controller are provided on the operating handle, the controller being configured to perform data processing on the health information of the object, and sending the health information of the object to the display device for displaying.

7. The endoscope auxiliary device according to claim 6, wherein the controller is configured to trigger the display device to display an alarm when the health information of the object does not meet predetermined conditions.

8. The endoscope auxiliary device according to claim 6, wherein an alarm is further provided on the operating handle, the controller being configured to trigger the alarm to give an alarm when the health information of the object does not meet predetermined conditions.

9. The endoscope auxiliary device according to claim 6, wherein the health information collecting probe includes a temperature collecting probe, a humidity collecting probe or a humiture collecting probe.

10. The endoscope auxiliary device according to claim 6, wherein an image collecting device is further provided on an end of the probing body away from the operating handle; and
the controller is further signally communicated with the image collecting device, the controller being configured to perform data processing on the image collected by the image collecting device, and sending the image to the display device for displaying.

11. The endoscope auxiliary device according to claim 6, wherein at least one of a bluetooth transmission device, a USB interface and a video transmission interface is further provided on the operating handle.

12. The endoscope auxiliary device according to claim 2, wherein a magnifying device is provided on an end of the probing body away from the operating handle.

13. The endoscope auxiliary device according to claim 2, wherein the light source includes a light-emitting diode, and the probing body includes a probing body made of material of polymethyl methacrylate.

14. The endoscope auxiliary device according to claim 3, wherein the light source includes a light-emitting diode, and the probing body includes a probing body made of material of polymethyl methacrylate.

15. The endoscope auxiliary device according to claim 2, wherein the endoscope auxiliary device comprises an endoscope auxiliary device for nasal cavity, an endoscope auxiliary device for ear canal, a laryngendoscope auxiliary device, or an endoscope auxiliary device for mouth.

16. The endoscope auxiliary device according to claim 3, wherein the endoscope auxiliary device comprises an endoscope auxiliary device for nasal cavity, an endoscope auxiliary device for ear canal, a laryngendoscope auxiliary device, or an endoscope auxiliary device for mouth.

17. The endoscope auxiliary device according to claim 2, wherein,
a health information collecting probe configured to collect health information of an object is provided on an end of the probing body away from the operating handle; and
a controller signally communicated with the health information collecting probe and a display device signally communicated with the controller are provided on the operating handle, the controller being configured to perform data processing on the health information of the object, and sending the health information of the object to the display device for displaying.

18. The endoscope auxiliary device according to claim 3, wherein, p1 a health information collecting probe configured to collect health information of an object is provided on an end of the probing body away from the operating handle; and
a controller signally communicated with the health information collecting probe and a display device signally communicated with the controller are provided on the operating handle, the controller being configured to perform data processing on the health information of the object, and sending the health information of the object to the display device for displaying.

19. The endoscope auxiliary device according to claim 2, wherein the light source includes a light-emitting diode, and the probing body includes a probing body made of material of polymethyl methacrylate.

20. The endoscope auxiliary device according to claim 8, wherein the health information collecting probe includes a temperature collecting probe, a humidity collecting probe or a humiture collecting probe.
